(19) United States
(12) Patent Application Publication
(10) Pub. No.: US 2010/0233112 A1
(43) Pub. Date: Sep. 16, 2010

(54) SHAPE MEMORY POLYMER NETWORK USING HETEROCYCLIC GROUPS

(76) Inventors: Jinlian Hu, Hong Kong (CN); Shaojun Chen, Hong Kong (CN)

Correspondence Address: WILKINSON & GRIST, 6TH FLOOR, PRINCE''S BUILDING, CHATER ROAD, CENTRAL, HONG KONG, HK

(21) Appl. No.: 12/687,896

(22) Filed: Jan. 15, 2010

Related U.S. Application Data
(60) Provisional application No. 61/145,519, filed on Jan. 16, 2009.

(51) Int. Cl. A61K 8/87; C08G 18/00; C08F 26/06; A61Q 5/00; C08J 9/00

(52) U.S. Cl. 424/70.11; 528/65; 526/263; 521/159; 521/147

(57) ABSTRACT

A shape memory polymer network may include at least one polymer, a plurality of proton accepting heterocyclic groups, a plurality of proton donating groups, and a plurality of cross-link moieties. At least one of the proton accepting heterocyclic groups and the proton donating groups is attached to the at least one polymer. The network may include an original shape at zero stress, and may include a deformed shape upon subjection to a stress. The network changes from the deformed shape to the original shape upon exposure to an external stimulus.

This application claim benefit from U.S. Provisional Patent Application No. 61/145,519.

### BACKGROUND

Shape memory materials have drawn wide attention because of their ability to recover their original shapes upon exposure to an external stimulus. Shape memory materials can find applications in sensors, actuators, smart devices, and media recorders. Examples of shape memory materials may include shape memory alloys, shape memory ceramics, and shape memory polymers.

Traditional shape memory polymers use elastic polymer networks that are equipped with stimuli-sensitive switches. The driving force for shape recovery in these shape memory polymers has usually been the elastic strain that is generated by deformation or by raising the surrounding temperature above the response temperature of the polymers. Deformation at high temperature is easier to achieve due to the low rubbery modulus of the polymers that may make the orientation of the polymer more feasible. However, the orientation may become partly relaxed before the structure can be frozen during subsequent cooling cycles. On the other hand, deformation at low temperature is difficult due to the high glassy state modulus of the polymers.

It is therefore desirable to develop a novel shape memory polymer network with improved physical and chemical properties as well as composition formulations. It is also desirable to develop a shape memory polymer network that can exhibit a good shape memory effect.

### BRIEF SUMMARY

According to one aspect, a shape memory polymer network may include at least one polymer, a plurality of proton accepting heterocyclic groups, a plurality of proton donating groups, and a plurality of cross-link moieties. At least one of the proton accepting heterocyclic groups and the proton donating groups is attached to the at least one polymer. The network may include an original shape at zero stress, and may include a deformed shape upon subjection to a stress. The network changes from the deformed shape to the original shape upon exposure to an external stimulus.

### BRIEF DESCRIPTION OF THE DRAWINGS

FIG. 1A depicts the structure of a supramolecular SMP network including pyridine moieties in a side-chain.

FIG. 1B depicts the structure of a supramolecular SMP network including pyridine moieties in a main-chain.

FIG. 2 depicts the mechanical properties of the side-chain PUPy series samples.

FIG. 3 depicts the thermal-sensitive strain-stress curves of a side-chain PUPy-MDI sample.

FIG. 4 depicts the moisture-sensitive recovery curves of the side-chain PUPy samples.

FIG. 5 depicts the mechanical properties of a main-chain PUPyB sample.

FIG. 6 depicts the thermal-sensitive strain-stress curves of a main-chain PUPyB sample.

FIG. 7 depicts the thermal-sensitive strain-stress curves of PUPyA/PAA polymer blends.

### DETAILED DESCRIPTION

Reference will now be made in detail to a particular embodiment of the invention, examples of which are also provided in the following description. Exemplary embodiments of the invention are described in detail, although it will be apparent to those skilled in the relevant art that some features that are not particularly important to an understanding of the invention may not be shown for the sake of clarity.

Furthermore, it should be understood that the invention is not limited to the precise embodiments described below, and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims. In addition, improvements and modifications which may become apparent to persons of ordinary skill in the art after reading this disclosure, the drawings, and the appended claims are deemed within the spirit and scope of the present invention.

A shape memory polymer network may include at least one polymer, a plurality of proton accepting heterocyclic groups, a plurality of proton donating groups, and a plurality of cross-link moieties. At least one of the proton accepting heterocyclic groups and the proton donating groups is attached to the at least one polymer. The network may include an original shape at zero stress, and may include a deformed shape upon subjection to a stress. The network changes from the deformed shape to the original shape upon exposure to an external stimulus. The proton accepting heterocyclic groups, the proton donating groups, and the cross-link moieties may account for from 10 to 90 weight percent of the network.

Composition

The proton accepting heterocyclic groups of the network may include at least one pyridine moiety. The pyridine moieties may be formed from a pyridine selecting from the group consisting of ABT-089, abiraterone, 4-aminopyridine, 3,4-diaminopyridine, anabasine, bipyridines, clopyralid, collidine, dianicline, difenpiramide, epibatidine, esomeprazole, fusaric acid, GTS-21, gapicomine, 3-hydroxy picolinic acid, imidacloprid, iproniazid, isoniazid, isonicotinamide, isonicotinic acid, ispronicline, lansoprazole, lercanidipine, linopirdine, 2,6-lutidine, mepyramine, methapyrilene, methylpyridine, milrinone, niacin, nialamide, niceritrol, nicofuranose, nicorandil, nicotinamide, nicotine, nicotinyl alcohol, niflumic acid, nikethamide, nitenpyram, N-nitrosonornicotine, orellanine, picotamide, pirbuterol, pirisudanol, polypyridine complexes, protionamide, pyridoxal, pyridoxamine, pyriproxyfen, pyritinol, quinolinic acid, risedronic acid, rivanicline, rosiglitazone, SCH-530348, sulfapyridine, sulfasalazine, sulfur trioxide pyridine complex, taranabant, tebanicline, triclopyr, and derivatives thereof.

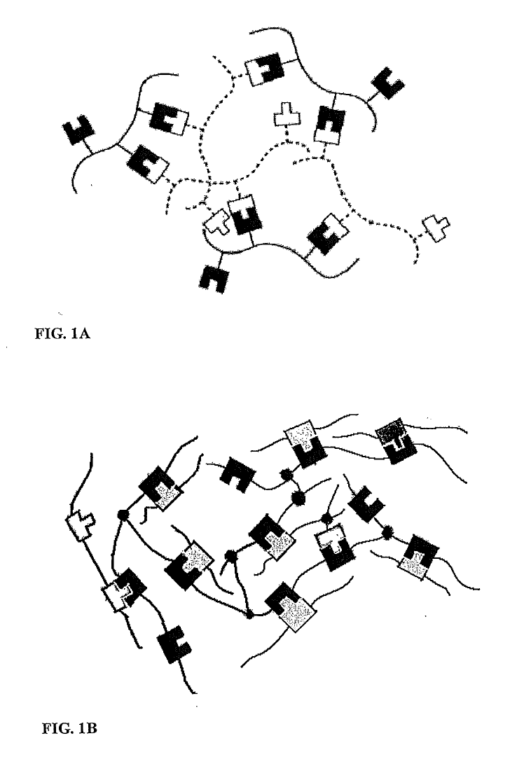

If the pyridine moieties are attached to the polymer of the network, they may be connected to the side chains of the polymer, such as depicted in FIG. 1A, and/or they may be part of the main chain of the polymer, such as depicted in FIG. 1B. The pyridine moiety may be connected to an intermolecular chain of the polymer. The polymer may be formed by polymerizing monomers that include a pyridine moiety. Examples of monomers containing pyridine moieties include N,N-bis(2-hydroxyethyl) isonicotinamine (BINA); 2,6-bis(hydroxymethyl)pyridine (HMP); 2,6-diaminopyridine; and 2,6-pyridine dicarboxylic acid. Other 2,6- and 2,5-pyridine units known to one of ordinary skill in the art may also be included.

The proton accepting heterocyclic groups may include at least one moiety formed from a heterocycle selected from the group consisting of furan, pyridazine, pyrazine, selenophene, oxazole, indole, imidazole, pyran, pyrimidine, pyrazine, pyrazole, pyrrole, thiopyran, thiophene, tellurophene, and derivatives thereof.

The proton donating groups of the network may act as proton—donors and may provide protons to form hydrogen bonding with the proton accepting heterocyclic groups. The proton donating groups may include at least one group selected from the group consisting of —NH, such as —NH of urethane group; —NH₂; —NH₃; —NH₄; phenol; aliphatic alcohol, such as —OH of hydroxyethyl acrylate and —OH of hydroxy-cellulose; carboxylic acid, such as —COOH of polyacrylic acid; and sulfuric acid.

The cross-link moieties of the network may form physical net-points or chemical cross-linking net-points. The term “net-point” means domains of the polymer that relate to the highest thermal transition temperature. The physical net-points may be formed from the group consisting of a phenyl group, a heterocyclic group, a crown ether group, a polar group, and a urethane group. The chemical cross-linking net-points may be formed from the group consisting of a carbon-carbon double bond group, a hydroxyl group, a carboxylic acid group, an isocyanate group, and an acyl halide group. The acyl halide group may include acyl chloride or acyl bromide.

The shape memory polymer network may also include a segment configured to control and adjust the shape changing conditions of the network, and/or to adjust the dissolvability and the mechanical properties of the network. The segment may be attached to the main-chain of the polymer, to the side-chain of the polymer, or to another polymer chain. The segment may be selected from a group consisting of a polyether group, an alkyl chain group, a hydrophobic group, and a hydrophilic group. Other groups known to one of ordinary skill in the art may also be included. The conditions to be controlled or adjusted may include a response temperature, a response concentration of a gas, and a response speed.

Method

The network may be formed from a shape memory polymer chain that may include a backbone selected from the group consisting of a carbon chain, a carbon-oxygen chain, a silicon chain, a silicon-oxygen chain, and mixtures thereof. The shape memory polymer may be selected from the group consisting of graft polymers, linear polymers, and dendrimer polymers. Other types of polymers known to one of ordinary skill in the art may also be included.

The shape memory polymer network may include homopolymers, copolymers, interpenetrating networks, semi-interpenetrating networks, and polymer blends. Other types of polymer network known to one of ordinary skill in the art may also be included.

A method of preparing the shape memory polymer network may include polymerizing monomers containing the proton accepting heterocyclic groups, the proton donating groups, and/or the cross-link moieties using a technique that may include a free radical polymerization method, an ionic polymerization method, a condensation polymerization method, a coordination polymerization method, or an atom transfer radical polymerization method. Conventional processing techniques such as extrusion, injection, blow molding, and laser ablation may be also used to manufacture the polymer network. Other techniques known to one of ordinary skill in the art may also be used.

Separation Structure

The shape memory polymer network may form a phase separation structure. The phase separation structure may include at least one switching phase and at least one kind of net-point. The switching phase may have a high fraction of reversible hydrogen bonding. The net-point may include a physical cross-linking net-point through non-covalent bonding, or may include a chemical cross-linking net-point through covalent bonding or reversible covalent bonding.

The dissociation temperature of the switching phase may be lower than that of the physical net-points, and may range from −50° C. to 200° C. The physical cross-linking net-points may be related to a higher dissociation temperature of the non-covalent bonding, and may range from −20° C. to 250° C. The chemical cross-linking net-points based on reversible covalent bonding may be related to a reversible association-disassociation temperature of at least 30° C. higher than the dissociation temperature of switching segment (e.g. Tₜᵣₐₙₛ+30° C.), and may range from −20° C. to 250° C.

Phase separation morphology may be obtained by anneal training at a temperature ranging from 50° C. to 120° C. Phase separation morphology may be obtained by a polymer coagulation technology that may include deposition, and/or applying low temperature or pressure. In one example, the polymer compositions may be put in an oven of 100° C. for 12 hours, and may then be cooled down to room temperature for 12 hours. In another example, a polymer film may be cast onto polytetrafluoroethylene (PTFE) by polymer solutions in dimethyl formamide (DMF), which may be washed slowly several times with water or ethanol. The film may be dried at room temperature under a flow or in a vacuum oven to obtain good phase separation morphology.

Recovery

An original shape of the shape memory polymer network may be manufactured from the shape memory polymers described above. A deformed shape of the network may be made through extension, compression, bending, and/or pressure molding, after the material has been softened (i.e. at a lower modulus). Moreover, the polymer may be deformed by subjection to a stress. Examples of stresses may include heating the polymer to a temperature above the response temperature (Tₜᵣₐₙₛ), and/or immersing the polymer in water, moisture, or a chemistry gas. The response temperature may be a centered temperature at which the modulus of materials may decrease significantly. The response temperature may be related to the dissociation temperature of non-covalent bonding, such as hydrogen-bonding, at a switching segment, to the glass transition temperature (T_{g}) of the polymer chain, or to the crystal melting temperature (Tₘ) of a switching segment. The Tₜᵣₐₙₛ may range from −40° C. to 200° C.

The deformed shape memory polymer network may recover its original shape upon exposure to an external stimulus. The external stimulus may be selected from the group consisting of heat, light, electricity, magnetic fields, ultrasound, liquid water, water vapor, acetic acid in the form of a gas or liquid, and combinations thereof. Other stimuli known to one of ordinary skill in the art may also be included.

The deformed shape memory polymer network may recover its original shape upon exposure to another external stimulus. The polymeric materials of the network may be stiffened as the modulus increases upon exposure to another external stimulus. Examples of the external stimulus include cooling the surrounding temperature to below the Tₜᵣₐₙₛ, drying the polymer network at a temperature of from 40 to 100° C. and at a condition with relative humidity of less than 40%, and deformation under an external force of greater than 0.5 MPa. Other stimuli known to one of ordinary skill in the art may also be included.

The permanent shape of the shape memory polymer network may be recovered when the surrounding temperature is higher than Tₜᵣₐₙₛ of the shape memory polymer network. The permanent shape of the shape memory polymer network may be recovered when the Tₜᵣₐₙₛ of the shape memory polymer is reduced to a temperature below the surrounding temperature, after the polymer networks have been immersed in water, moisture, or a chemistry gas or solvent. The Tₜᵣₐₙₛ may range from −40° C. to 200° C.

The response relative humidity is defined as the relative humidity at which the modulus value of a polymeric material decreases, after being immersed in the external stimulus during the response time. The response time is defined as the time at which the temporary shape of the polymer network recovers 90% of the maximum recovery. The response time may range from 1 minute to 48 hours. The response relative humidity may range from 40% to 100%. The response relative chemical gas concentration is defined as the relative chemical gas concentration at which the modulus value of the polymeric material decreases, after being immersed in the external stimulus during which the response time is at the range of 40% to 100%.

Applications

The shape memory polymer networks may be used in combination with other materials, whether or not the other materials have the shape memory effect. In one example, a poly(acrylic acid) may be developed into a shape memory polymer composite by blending with at least 30 weight percent of poly(vinylpyridine-co-methylacrylate). In another example, shape memory liquid crystalline polymer may be achieved in the complex of shape memory polyurethanes containing pyridine moieties with DOBA (4-dodecyloxybenzoic acid).

The shape memory polymer network may be used in a wide range of applications, from aerospace to civil engineering, and to domestic products in the form of solution, gel, film, fiber, board, composite, and foam. In one example, the shape memory polymer solution or gel can be adhered on the hair of humans or animals, and hair having shape memory effect may be achieved after the evaporation of the solvent in the solution or gel. In another example, shape memory polymer fiber may be spun by wet-spinning or melt-spinning from the polymer solution or bulk polymer chips. A shape memory fabric may be fabricated using the shape memory polymer fiber in combination with cotton or wool. Textiles having the shape memory polymer fiber may then be made from the fabric.

Having described embodiments of the present system with reference to the accompanying drawings, it is to be understood that the present system is not limited to the precise embodiments, and that various changes and modifications may be effected therein by one having ordinary skill in the art without departing from the scope or spirit as defined in the appended claims.

Furthermore, it should be understood that the polymer network is not limited to the precise embodiments described below and that various changes and modifications thereof may be effected by one skilled in the art without departing from the spirit or scope of the invention. For example, elements and/or features of different illustrative embodiments may be combined with each other and/or substituted for each other within the scope of this disclosure and appended claims.

### EXAMPLES

### Example 1

### Preparation of Shape Memory Polyurethane Containing Pyridine Moieties (PUPy) in a Side Chain

A pyridine derivative, N,N-bis(2-hydroxylethyl) isonicotinamine (BINA), was used to prepare a polyurethane with hexamethylene diisocyanate (HDI). A series of PUPy samples were synthesized with the addition of 1,4-butanediol (BDO) and/or methylene diphenyl diisocyanate (MDI). The compositions of the samples with different content of BINA are presented in Table 1. The molecular structure of PUPyA is presented in Scheme 1. The molecular structure of PUPy-MDI is presented in Scheme 2.

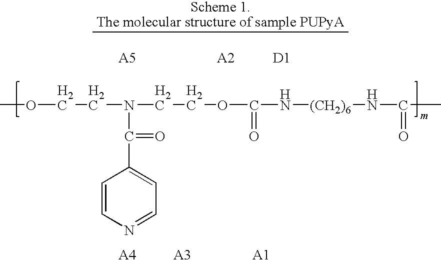

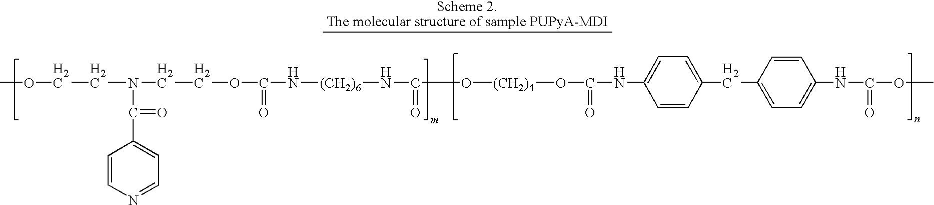

TABLE 1
The composition of side-chain shape memory polyurethane

| | BINA | HDI | BDO | MDI | Content of |
|---|---|---|---|---|---|
| samples | (g) | (g) | (g) | (g) | BINA (wt %) |
| PUPyA | 10 | 8.0 | — | — | 55.6 |
| PUPy-BDO | 3.0 | 5.0 | 1.4 | — | 31.9 |
| PUPy-MDI | 4.0 | 5.8 | 2.1 | 2.0 | 28.8 |

The reaction was carried out at 80° C. for 2 hours in a 500-mL, round-bottom, four-necked flask filled with nitrogen and equipped with a mechanical stirrer, a thermal meter, and a condenser to prepare the pre-polymer with BINA and HDI. For the PUPy-BDO and PUPy-MDI samples, BDO and MDI were added to the pre-polymer of HDI-BIN for another 2 hours. During the reaction process, dimethylformamide (DMF) was added into the reaction to occasionally control the viscosity of the solution. Thereafter, 10.0 weight percent of PU/DMF solution was poured out from the flask for film casting. The samples could be prepared after the films were placed at 60° C. for 24 hours and further dried at 75° C. under vacuum of from 0.1 to 0.2 KPa for 24 hours.

### Example 2

### Properties of Shape Memory Polyurethane Containing Pyridine Moieties (PUPy) in a Side Chain

From the structure of PUPyA, the prepared shape memory polyurethane was observed to include a pyridine ring attached to the backbone of polyurethane chain as a pendent. The shape memory polyurethane was also observed to include a —NH at the urethane group that acted as proton-donors. The proton donor (D1) formed a strong hydrogen bonding with the nitrogen of the pyridine ring (A4), and also formed other strong hydrogen bonding with the C═O of the urethane group, as seen in Scheme 1. Other types of hydrogen bonding formed between D1 and A2, A3, and A5.

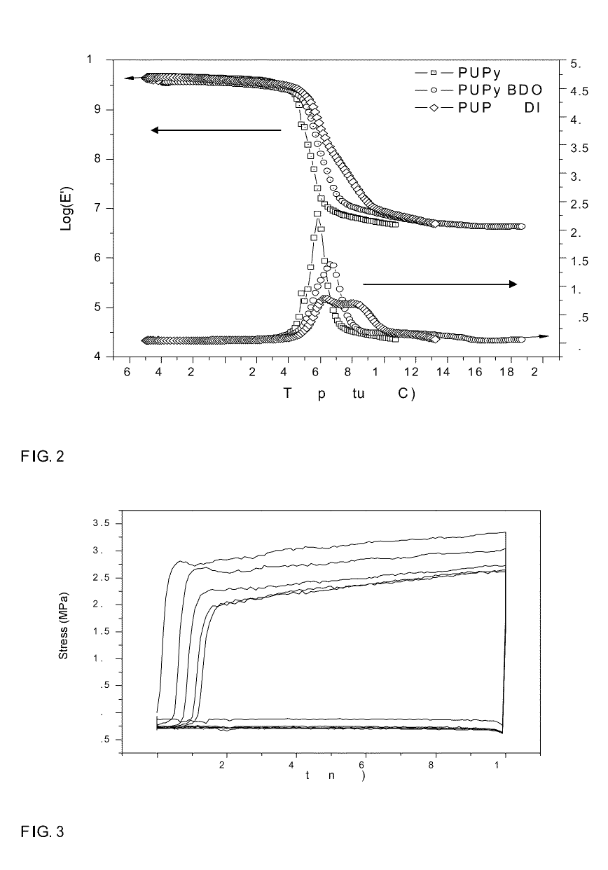

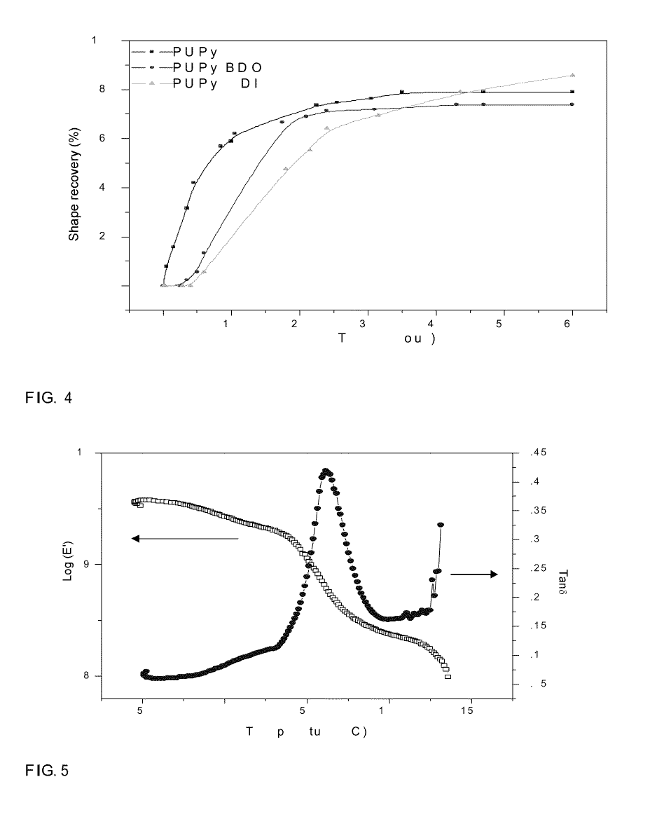

In the PUPy-MDI system, there may exist dipole-dipole interaction between the phenyl groups of MDI, to enable the domains or net-points with high dissociation temperature to be formed at the urethane groups. Particularly in the PUPy-MDI system, other hydrogen bonds may also influence the mobility of the polymer chain. The dissociation of hydrogen bonding, for example the D1 to A4 hydrogen bonding, significantly decreased the modulus, as depicted in FIG. 2. Consequently, the thermoplastic polyurethanes have shown excellent shape memory effects, such as thermo-sensitive shape memory effects, as depicted in FIG. 3, water/moisture-sensitive shape memory effects, as depicted in FIG. 4, and chemical gas-sensitive shape memory effects, as shown in Table 2.

The dynamical mechanical properties of polyurethanes containing pyridine moieties are depicted in FIG. 2. A significant decrease in modulus was observed at about 60° C. with a desirable modulus ratio (Eg/Er>800). Eg is defined as the glassy state modulus, and Er is defined as the rubbery modulus. High shape fixity near 100% and a high shape recovery above 95% were observed for the polyurethane, as depicted in FIG. 3. The results were attributed to phase separation structure of the polyurethane, as discussed above.

Moreover, the supramolecular shape memory polyurethane responded to moisture and water. High water/moisture absorbability was observed in the polyurethane containing pyridine moieties, since the strength of the hydrogen bonding may depend on parameters such as temperature, pressure, bond angle, and environment. This resulted in the dissociation of hydrogen bonding, accompanied by a decrease in the glassy modulus.

The PUPyA sample started to recover its deformed strain after it had been immersed in the moisture (RH=60%) for 0.5 hours, and the sample recovered 60% of the deformed strain in 10 hours, as depicted in FIG. 4. When the content of pyridine moieites decreased, the time at which the sample began to recover was delayed to 2.5 hours for PUPy-BDO sample and to 7.5 hours for PUPy-MDI sample.

Similarly, the supramolecular shape memory polyurethane containing pyridine moieties also responsed to acetic acid gas, as shown in Table 2. Acetic acid provided protons that formed strong hydrogen bondings with the nitrogen of the pyridine rings. This decreased the glassy modulus at room temperature. Thus, the deformed sample recovered its original shape with the stimulus of acetic acid gas. In Table 2, PUPy-BDO and PUPy-MDI started to recover after they had been immersed in the acetic acid gas for 1 hour and 1.5 hours, respectively. These samples finished the recovery after 9 hours and 6 hours, respectively. Due to the stable physical netpoint, close to 90% of shape recovery was achieved in the PUPy-MDI sample, and close to 62% of shape recovery was ahcieved in the PUPy-BDO sample. However, the BINA-HDI alternative copolymer was dissolved by acetic acid gas because of their weak non-covalent interaction of physical net-points.

TABLE 2
The strain recovery of shape memory polyurethane in acetic acid gas

| | Length at | Length at | Time when | Time when | |
|---|---|---|---|---|---|
| | start of | end of | recovery | recovery | Recovery |
| | recovery, | recovery, | began, | finished, | Rate, |
| Sample | Lₛₜₐᵣₜ (mm) | L_{end} (mm) | Tₛₜₐᵣₜ (h) | T_{final} (h) | Rᵣ (%) |
| PUPyA | 47.0 | 47.0 | — | — | — |
| PUPy-BDO | 42.0 | 28.0 | 1.0 | 9.0 | 61.9 |
| PUPy-MDI | 38.0 | 22.0 | 1.5 | 6.0 | 88.89 |

### Example 3

### Application of Shape Memory Polyurethane Containing Pyridine Moieties (PUPy) in a Side Chain on the Hair Showing Shape Memory Effect

The solvent was removed from the shape memory polyurethanes as prepared according to Example 1. The solid products of shape memory polyurethane were then dissolved in a benign solvent such as acetic acid or ethanol to prepare a 3 to 20 wt % mixture. The mixture was then sprayed equally on dry hair. Due to the strong hydrogen bonding between the polymer and hair, the shape memory polyurethane covered the surface of hair stably after the solvent was evaporated at room temperature or by a hair drier.

Subsequently, the hair in a straight shape covered with shape memory polyurethanes was heated to 70° C., which was above the dissociation temperature of the hydrogen bonding for the polymer. The straight hair was fabricated into a curl-like shape, although other shapes or styles are also envisioned. The shape of the hair was fixed after the hair cooled down to room temperature. The shape memory polymer enabled the shape or style of the hair to be maintained.

Finally, to recover the original shape or style of the hair, the temporary hair shape or hair style was heated up to 70° C. again using the hair drier. The shape memory polymer enabled the hair to recover the original shape or style because of the shape recovery of shape memory polymer and its higher shape recovery force. Table 3 summarizes the testing results on hair finishing with the shape memory polymers.

TABLE 3
The testing results of hair finishing
with the shape memory polyurethane

| | Polymer | Polymer | Shape | shape |
|---|---|---|---|---|
| | solution | weight | fixity | recovery |
| Sample | in acetic acid (%) | on hair (g) | of hair (%) | of hair (%) |
| PUPy-MDI | 5% | 0.06 | 95 | 82 |
| PUPy-BDO | 5% | 0.06 | 94 | 75 |
| PUPyB | 5% | 0.06 | 97 | 90 |

### Example 4

### Preparation of Shape Memory Polyurethane Containing Pyridine Moieties in Main Chain

In this example, 2,6-bis(hydroxymethyl)pyridine (HMP) was used to prepare the polyurethane with the hexamethylene diisocyanate (HDI). A series of PUPy samples were synthesized with the addition of diethylene glycol (DEG) and/or IPDI (isophorone diisocyanate). The compositions of the samples with different content of HMP are presented in Table 4.

TABLE 4
The composition of the main-chain shape memory polyurethane

| | HMP | HDI | DEG | IPDI | Content of |
|---|---|---|---|---|---|
| samples | (g) | (g) | (g) | (g) | HMP (wt %) |
| PUPyB | 10 | 12 | — | — | 45.5 |
| PUPyB-DEG | 8 | 12 | 1.9 | — | 36.5 |
| TuPyB-IPDI | 8 | 12 | 3.1 | 2.0 | 31.8 |

The reaction was carried out at 80° C. for 2 hours in a 500-mL, round-bottom, four-necked flask filled with nitrogen and equipped with a mechanical stirrer, a thermal meter, and a condenser to prepare the pre-polymer with HMP and HDI. For the PUPy-DEG and PUPy-IPDI samples, DEG and IPDI were added to the pre-polymer of HDI-HMP for another 2 hours. During the reaction process, DMF was added into the reaction to occasionally control the viscosity of the solution. Then, 10 weight percent of the polyurethane-DMF solution was poured from the flask for film casting. The samples were prepared after the films were placed at 60° C. for 24 hours and further dried at 75° C. under vacuum of from 0.1 to 0.2 KPa for 24 hours. The pyridine ring was connected to backbone of polyurethane, as shown in Scheme 3.

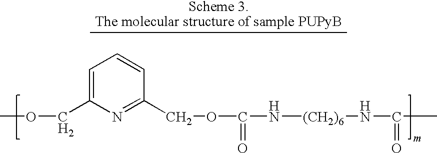

### Example 5

### Properties of Shape Memory Polyurethane Containing Pyridine Moieties in Main Chain

The sample contained proton-acceptors at the pyridine ring and proton-donors at the urethane groups. More than two kinds of hydrogen bonding were formed between the pyridine rings and the urethane groups. Since the urethane group acted as the netpoint, the pyridine ring existed in the switching phase, and the hydrogen bonding influenced the movement of polyurethane chain. A significant modulus decrease was found in its DMA curves, as shown in FIG. 5. The responsive temperature ranged from 60° C. to 75° C. However, when compared with the side-chain polyurethane based on BINA as reported in Example 2, the HMP based polyurethane had about 100 times the modulus ratio (Eg/Er≈100).

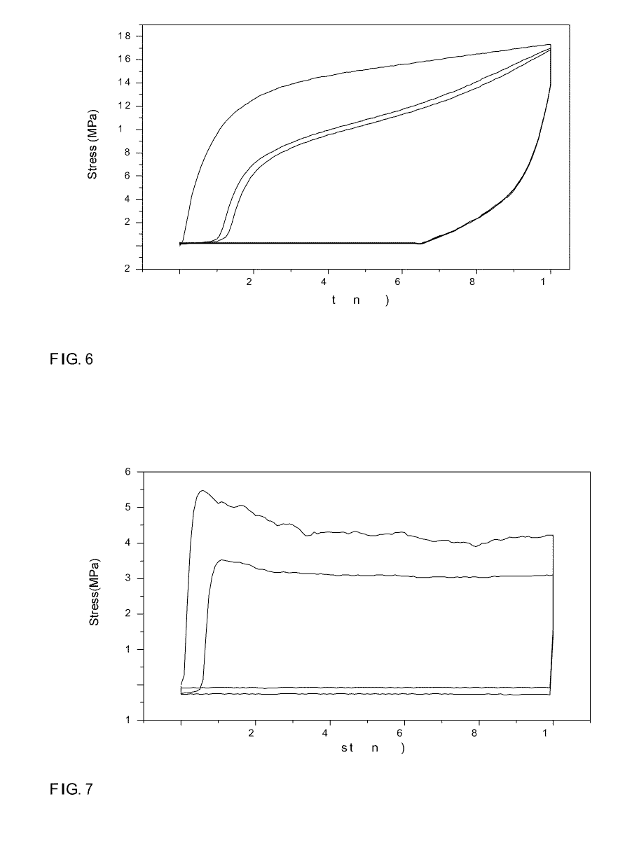

The thermal-induced shape memory effect was achieved. The strain-stress curves are presented in FIG. 6. The shape fixity ranged from 60% to 90%, and shape recovery ranged from 80% to 95%. The shape memory behavior may be modified with the addition of DEG and IPDI, and the shape recovery temperature can be adjusted by controlling the HMP content.

### Example 6

### Shape Memory Polyurethane/PAA-co-BA Copolymer Blends

In this example, a polymer blend including a shape memory polyurethane with a poly(acrylic acid-co-butylacrylate) PAA-co-BA copolymer was prepared. The shape memory polyurethane PUPyA was first synthesized according to the procedure given in Example 1. The PAA-co-BA copolymers were synthesized by a free radical polymerization method with a ratio of 1:2. Then, 10 g of PUPyA polymer and 10 g of PAA-co-BA polymer were dissolved into the same DMF mixture and stirred mechanically for 24 hours. A film of the PUPyA/PAA-co-BA polymer blend was prepared after the DMF evaporated at 100° C. for 24 hours.

The PAA-co-BA copolymer of the polymer blend contained carboxylic acid groups acting as proton-donors. Strong hydrogen bonding was formed between the nitrogen of the pyridine ring and the carboxylic acid of the PAA-co-BA, which restricted the movement of the PU chains and the PAA-co-BA chains. The reversible dissociation-association of hydrogen bonding resulted in the glassy modulus decreasing significantly. The interaction of the urethane group of PUPy or the semi-IPN net-points of polymer blends formed the physical netpoint of the polymer blends.

Cyclic tensile tests were performed to investigate the shape memory behavior of PUPyA/PAA-co-BA polymer blends. The strain-stress curves of the polymer blends are presented in FIG. 7. Excellent shape memory behavior was achieved in the polymer blends. The shape fixity was close to 100%, and the shape recovery was above 97%. The shape recovery temperature was about 75° C., higher than that of the PUPyA sample. Moreover, the polymer blends had a higher glassy modulus and a higher maximum tans than that of the pure PUPyA sample, since the PUPyA/PAA-co-BA polymer blends contained stronger hydrogen bonds.

### Example 7

### Cross-Linked Shape Memory PVP/TA Complex

In this example, a cross-linked complex using poly(4-vinylpyridine) PVP with TA (terephthalic acid) was prepared to exhibit the shape memory effect.

The cross-linked PVP/TA complex was prepared using the following procedure and according to the composition as shown in Table 5. For the PVP-EA/TA sample, 10 g of 4-vinylpyridine (VP), 4 g of 2-hydroxyethyl acrylate (HEA), 1 g of ethylene glycol dimethacrylate (EGDA), and 5 g of TA were added to a 500-mL, round-bottom, four-necked flask filled with nitrogen and equipped with a mechanical stirrer, a thermal meter, and a condenser. The reaction mixture was initialized with 0.01 g of AIBN in 30 ml of DMF at 80° C. and was kept in the free radical polymerization for 48 hours. The reaction mixture was cooled down to room temperature, and 2 g of MDI as cross-linker was added to the slightly cross-linked PVP/TA DMF solution. The mixture solution was poured into a PTFE flat after stirring the mixture for 30 minutes by mechanical stirring for homogenous mixing. Finally, the PVP/TA polymer complex film was prepared after the polymer film was further cross-linked and annealed, accompanied by DMF evaporation, in a 100° C. oven for 24 hours.

TABLE 5
The composition of crosslinked shape memory PVP/TA complex

| | VP | EGDA | TA | AIBN | HEA | AA | MDI |
|---|---|---|---|---|---|---|---|
| samples | (g) | (g) | (g) | (g) | (g) | (g) | (g) |
| PVP/TA | 10.0 | 2.0 | 5.0 | 0.01 | — | — | — |
| PVP-EA/TA | 10.0 | 1.0 | 5.0 | 0.01 | 4.0 | — | 2.0 |
| PVP-AA/TA | 10.0 | 2.0 | 5.0 | 0.01 | — | 4.0 | 1.0 |

In the PVP/TA shape memory complex, hydrogen bonding was formed between the nitrogen of PVP and the carboxylic acid of TA, acting as the reversible switch. The chemical cross-linking included the EGDA segment and HEA-MDI segment, acting as the net-point. The dissociation-association transition of hydrogen bonding resulted in the following shape memory effect: a shape fixity of more than 95%, a shape recovery of more than 85%, and a shape recovery temperature of about 60-80° C. Moreover, the shape memory effect was adjustable by controlling the density of cross-linking and by controlling the relative amounts of PVP, TA and HEA.

### Example 8

### Shape Memory Cellulose and Fibers

Cellulose is believed to contain a plurality of hydroxyl groups, and can be modified to contain carboxylic acid groups. In this example, celluloses containing both hydroxyl and carboxylic acid groups were selected from carboxymethyl cellulose (CMC) with Mn>100,000.

The shape memory cellulose was prepared with the following procedures: 10 g of CMC were dissolved with 100 g of DMF solvent. Then, 5 g of 4,4′-bipyridine (BPY) was added to the CMC/DMF solution. The temperature of mixture was increased to about 80° C. and was mixed for 48 hours with a mechanical stirrer. Finally, a homogenous BPY-CMC/DMF solution was obtained, and the BPY-CMC film was prepared by pouring the solution in PTFE and evaporating the DMF solution in a 100° C. oven for 24 hours.

While the polymer network has been described, it should be understood that the system is not so limited, and modifications may be made. The scope of the polymer network is defined by the appended claims, and all compositions that come within the meaning of the claims, either literally or by equivalence, are intended to be embraced therein.

What is claimed is:

1. A shape memory polymer network, comprising:
at least one polymer,
a plurality of proton accepting heterocyclic groups,
a plurality of proton donating groups, and
a plurality of cross-link moieties;
wherein at least one of the proton accepting heterocyclic groups and the proton donating groups is attached to the at least one polymer,
wherein the network comprises an original shape at zero stress, and comprises a deformed shape upon subjection to a stress, and
wherein the network changes from said deformed shape to said original shape upon exposure to an external stimulus.

2. The network of claim 1, wherein said proton accepting heterocyclic groups comprise at least one pyridine moiety.

3. The network of claim 2, wherein said pyridine moiety is formed from a pyridine selected from the group consisting of ABT-089, abiraterone, 4-aminopyridine, 3,4-diaminopyridine, anabasine, bipyridines, clopyralid, collidine, dianicline, difenpiramide, epibatidine, esomeprazole, fusaric acid, GTS-21, gapicomine, 3-hydroxy picolinic acid, imidacloprid, iproniazid, isoniazid, isonicotinamide, isonicotinic acid, ispronicline, lansoprazole, lercanidipine, linopirdine, 2,6-lutidine, mepyramine, methapyrilene, methylpyridine, milrinone, niacin, nialamide, niceritrol, nicofuranose, nicorandil, nicotinamide, nicotine, nicotinyl alcohol, niflumic acid, nikethamide, nitenpyram, N-nitrosonornicotine, orellanine, picotamide, pirbuterol, pirisudanol, polypyridine complexes, protionamide, pyridoxal, pyridoxamine, pyriproxyfen, pyritinol, quinolinic acid, risedronic acid, rivanicline, rosiglitazone, SCH-530348, sulfapyridine, sulfasalazine, sulfur trioxide pyridine complex, taranabant, tebanicline, and triclopyr.

4. The network of claim 3, where said pyridine moiety is connected to the side chains or to the main chain of the at least one polymer.

5. The network of claim 1, wherein said proton accepting heterocyclic groups comprise at least one moiety formed from a heterocycle selected from the group consisting of furan, pyridazine, pyrazine, selenophene, oxazole, indole, imidazole, pyran, pyrimidine, pyrazine, pyrazole, pyrrole, thiopyran, thiophene, and tellurophene.

6. The network of claim 1, wherein said proton donating groups comprise at least one group selected from the group consisting of —NH, —NH₂, —NH₃, —NH₄⁺, phenol, aliphatic alcohol, carboxylic acid and sulfuric acid.

7. The network of claim 1, wherein said cross-link moieties comprise physical cross-linking net-points formed from the group consisting of a phenyl group, a heterocyclic group, a crown ether group, a polar group, and a urethane group.

8. The network of claim 1, wherein said cross-link moieties comprise chemical cross-linking net-points formed from a group consisting of a carbon-carbon double bond group, a hydroxyl group, a carboxylic acid group, an isocyanate group, and an acyl halide group.

9. The network of claim 1, further comprising a segment formed from a group consisting of a polyether group, an alkyl chain group, a hydrophobic group, and a hydrophilic group.

10. The network of claim 1, wherein said stress comprises heating said polymer network to a temperature above a response temperature.

11. The network of claim 1, wherein said stress comprises immersing said polymer network in water, moisture, or a chemistry gas.

12. The network of claim 10, wherein said response temperature ranges from −40° C. to 200° C.

13. The network of claim 1, wherein said external stimulus comprises heat, light, electricity, magnetic field, ultrasound, water, or acetic acid.

14. The network of claim 1, wherein said external stimulus comprises cooling the polymer network to a temperature below a transition temperature of said network, drying said polymer network at a temperature of from 40 to 100° C. and at a condition with relative humidity of less than 40%, or deforming said network under an external force of greater than 0.5 MPa.

15. The network of claim 1, wherein said network is formed from homopolymers, copolymers, interpenetrating networks, semi-interpenetrating networks, polymer blends, or combinations thereof.

16. The network of claim 1, wherein said network is formed graft polymers, linear polymers, dendrimer polymers or combinations thereof.

17. The network of claim 1, wherein said network is in the form of a mixture, a gel, a film, a fiber, a board, a composite, or a foam.

18. The network of claim 1, further comprising a recovery rate of from 62% to 89%.

19. The network of claim 1, further comprising an initial recovery time of from 1 hour to 1.5 hours.

20. A hair sample, comprising a hair and the polymer network of claim 1, wherein said network comprises a shape fixity of from 94% to 97%, and a shape recovery of from 75% to 90%.
